SEQUENCE LISTING (2 sequences)

SEQ ID NO: 1 - protein, 76 aa, Homo sapiens [sequence omitted]

SEQ ID NO: 2 - protein, 16 aa, Artificial Sequence
  C-terminal peptide of probe
NIQKESTLHLVLRLRG

(19) United States
(12) Patent Application Publication
(10) Pub. No.: US 2021/0292736 A1
(43) Pub. Date: Sep. 23, 2021

(54) PROTEIN LABELLING

(71) Applicant: The Provost, Fellows, Scholars and other Members of Board of Trinity College Dublin, Dublin 2 (IE)

(72) Inventors: Joanna McGouran, Dublin 2 (IE); Neil Taylor, Dublin 2 (IE)

(21) Appl. No.: 17/167,366

(22) Filed: Feb. 4, 2021

(30) Foreign Application Priority Data
Mar. 18, 2020 (GB) 2003936.8

(51) Int. Cl. C12N 9/96; C12N 9/10; C07K 14/005; C12N 7/00; G01N 33/535

(52) U.S. Cl. CPC C12N 9/96; C12Y 203/02; C12N 9/104; C12N 2760/16022; C12N 7/00; G01N 33/535; C07K 14/005

(57) ABSTRACT

A process for labelling a target protein, such as a target enzyme, such as a deubiquitinating enzyme (DUB). The process comprises providing a probe-protein complex comprising a probe and the target protein. The probe comprises a recognition element and a warhead. The target protein comprises a cysteine residue and a recognition site. The recognition element is reversibly bound to the recognition site. A stimulus is applied to induce a radical reaction in the probe-protein complex to covalently bond the warhead to the cysteine residue, thereby labelling the target protein. This invention also resides in probes and probe-protein complexes for use in the process.

### CROSS REFERENCE TO RELATED APPLICATIONS

This application claims the benefit of priority under 35 U.S.C. § 119 of GB Application No. 2003936.8 filed Mar. 18, 2020, the contents of which are incorporated herein by reference in its entirety.

### FIELD

This invention relates to a process for labelling a protein (e.g. an enzyme) and probes and probe-protein complexes for use in the process.

### SEQUENCE LISTING

The contents of the electronic submission of the text file Sequence Listing, which is named BRB-41267.seq.listing_ST25(2).txt, which was created Apr. 30, 2021 and is 1.27 KB in size, is incorporated herein by reference in its entirety.

### BACKGROUND OF THE INVENTION

Ubiquitin is a 76 amino acid protein which is added post-translationally to protein substrates to modulate their activity and interactions. Ubiquitination is one of the most abundant post-translational modifications in eukaryotic cells, and is orchestrated by the widely conserved E1, E2 and E3 enzymes that sequentially activate, conjugate and ligate the ubiquitin monomers to substrate proteins.

Deubiquitinating enzymes (DUBs) possess ubiquitin C-terminal hydrolytic activity and are responsible for removal of ubiquitin from its conjugates. The human genome encodes for approximately 100 DUBs split into eight classes, seven of which are cysteine proteases. The repertoire of DUBs and conjugation machinery in eukaryotes, along with the differences in their relative promiscuity, result in highly complex enzymatic networks that regulate numerous critical cellular events. Disruption within these networks is associated with disorders including cancer, neurodegeneration and inflammation.

Activity-based protein profiling (ABPP) is a powerful chemical-proteomic strategy for the characterization of enzyme function in biological systems, which relies on the design of active-site directed covalently-binding probes to interrogate specific enzymes in complex environments. ABPs have been successful in identifying new DUB family members, DUB inhibitor assessment, characterisation of linkage selectivity and they have aided DUB crystallisation. Recent advances include cell permeable probes, methodologies to precisely identify labelling sites and probes resembling ubiquitin-substrate conjugates.

### SUMMARY OF THE INVENTION

According to a first aspect of the invention there is provided a process for labelling a target protein, the process comprising

providing a probe-protein complex comprising a probe and the target protein;
the probe comprising a recognition element and a warhead; and
the target protein comprising a cysteine residue and a recognition site;
wherein the recognition element is reversibly bound to the recognition site; and
applying a stimulus to induce a radical reaction in the probe-protein complex to covalently bond the warhead to the cysteine residue, thereby labelling the target protein.

In one embodiment, there is provided a process for labelling an enzyme, the process comprising

providing a probe-enzyme complex comprising a probe and the enzyme;
the probe comprising a recognition element and a warhead; and
the enzyme having a cysteine residue and a recognition site;
wherein the recognition element is reversibly bound to the recognition site; and
applying a stimulus to induce a radical reaction in the probe-enzyme complex to covalently bond the warhead to the cysteine residue, thereby labelling the enzyme.

The invention also resides in a probe-protein complex (e.g. probe-enzyme complex) for use in the process of the first aspect, a probe for use in the process of the first aspect and a labelled protein (e.g. a labelled enzyme) producible by the process of the first aspect.

The inventors have developed a ubiquitin-based activity based probe (ABP) that is inert under ambient conditions and can be activated upon application of a stimulus (e.g. exposure to UV light). As such, the probe can be viewed as latent until the stimulus is applied. The probe works via a radical mechanism within the enzyme active site. Without being bound by theory, the inventors submit that a primary radical source abstracts a hydrogen from the active site cysteine (RSH). The resulting thiyl radical (RS′) reacts with the aligned warhead (e.g. alkene moiety) of the probe affording a carbon-centred radical which likely abstracts a hydrogen from within the active-site. The result is a covalent bond between the probe (e.g. the C-terminus of the probe) and the active site cysteine. The active site is expected to remain accessible even post-binding. For example, DUBs typically act upon ubiquitinated substrates, leaving significant space proximal to the probe.

In existing probes, a reactive C-terminal moiety is aligned with the active site cysteine upon binding and a covalent bond is immediately formed via nucleophilic attack. In contrast, the process of the present invention requires the recognition element to be reversibly bound to the recognition site. This allows for the establishment of a binding equilibrium between the probe and the target protein (e.g. target enzyme) followed by a short activation period, allowing for temporal control which is not achievable using existing cysteine reactive probes (FIG. 11).

DUBs operate in several complex enzymatic networks and are known to be post translationally modified. Therefore, the ability to induce the formation of a probe-enzyme complex in vitro at specific times or after different external inputs offers the opportunity of a greater understanding of how these enzymes act within cells. This coupling reaction represents a promising and novel strategy to target the active form of these enzymes. Furthermore, this approach is readily applicable to a wide variety of other enzyme classes beyond the ubiquitin-proteasome system and cysteine proteases.

### Applying a Stimulus

Applying a stimulus to induce a radical reaction in the probe-protein complex (e.g. applying a stimulus to the probe-enzyme complex) may comprise exposing the probe-protein complex to light having a suitable wavelength and/or employing a free radical initiator and/or exposing the probe-enzyme complex to heat. Typically, applying a stimulus comprises exposing the probe-protein complex to light having a suitable wavelength and optionally employing a free radical initiator.

A light source may be employed to induce a radical reaction in the probe-enzyme complex. The reaction may be carried out in a photoreactor. Suitable light sources include LEDs and gas-discharge lamps.

The light may have a wavelength of 10 nm or more, 100 nm or more, 200 nm or more, 250 nm or more, 300 nm or more, 350 nm or more, 400 nm or more or 500 nm or more; and/or the light may have a wavelength of 700 nm or less, 600 nm or less, 500 nm or less, 400 nm or less, 300 nm or less or 200 nm or less. The light may have a wavelength of from 10 nm to 700 nm.

The light may have a wavelength of 10 to 400 nm. Light having this wavelength is typically described as ultraviolet light. The light may have a wavelength of 315 to 400 nm (UVA), 280 to 315 nm (UVB) or 100 to 280 nm (ultraviolet C).

The light may have a wavelength of 400 to 700 nm. Light having this wavelength is typically described as visible light. Visible light may be applied by exposure to ambient light or a light bulb (e.g. a conventional 10 AV light bulb).

The probe-protein complex may be degassed prior to exposure to the light, e.g. by degassing with nitrogen. The probe-protein complex may be degassed for 30 seconds or more, 1 minute or more or 2 minutes or more prior to exposure to the light.

The probe-protein complex may be exposed to the light (e.g. visible light) without prior degassing.

The probe-protein complex may be exposed to the light for a period of 60 minutes or less, 30 minutes or less, 20 minutes or less, 10 minutes or less, 5 minutes or less, 3 minutes or less 2 minutes or less or 1 minute or less; and/or the probe-enzyme complex may be exposed to the light for a period of 3 seconds or more, 5 seconds or more, 10 seconds or more, 30 seconds or more, 1 minute or more, 3 minutes or more or 5 minutes or more. It is an advantage of the present invention that the probe-protein complex need only be exposed to the light for a short period.

Applying a stimulus to induce a radical reaction in the probe-protein complex may comprise employing one or more radical initiators (also known as free radical initiators). A radical initiator is a substance that produces a radical species. The one or more radical initiators may be selected from azo compounds, halogens, acetophenones, and organic and inorganic peroxides for example.

Suitable free radical initiators include 2,2-dimethoxy-2-phenylacetophenone (DPAP), 2,2′-azobis(2-amidinopropane) dihydrochloride (AAPH), 4,4′-azobis(4-cyanopentanoic acid) (ACPA), tert-butylhydroperoxide, 4,4′-azobis(4-cyanovaleric acid), 1,1′-azobis(cyclohexanecarbonitrile), 2,2′-azobis (2-methylpropionamidine) dihydrochloride, 2,2′-azobis(2-methylpropionitrile), ammonium persulfate, hydroxymethanesulfinic acid, potassium persulfate, sodium persulfate, bismuth oxide, eosin (e.g. Eosin Y CAS 17372-87-1), cumene hydroperoxide, 2,5-di(tert-butylperoxy)-2,5-dimethyl-3-hexyne, dicumyl peroxide, 2,5-bis(tert-butylperoxy)-2,5-dimethylhexane, 2,4-pentanedione peroxide, 1,1-bis(tert-butylperoxy)cyclohexane, benzoyl peroxide, 9, 2-butanone peroxide, lauroyl peroxide, tert-butyl peroxybenzoate, tert-butylperoxy 2-ethylhexyl, 2,2′-azobisisobutyronitrile (AIBN), BPO (2-(4-Biphenyl)-5-phenyloxazole) and tert-butyl peracetate.

The one or more radical initiators may comprise an acetophenone and/or an azo compound and/or an organic peroxide. For example, the one or more radical initiators may comprise DPAP, AAPH, ACPA and/or tert-butylhydroperoxide. The inventors have determined that labelling can be carried out with each of these initiators, with DPAP being most successful. Applying a stimulus to induce a radical reaction in the probe-protein complex may comprise exposure to UV light together with the radical initiator DPAP.

The one or more free radical initiators may comprise bismuth oxide and/or eosin (e.g. Eosin Y). The inventors propose that these initiators would allow for a more gentle initiation process (e.g. in combination with visible light) making the process even more compatible with biological samples. If visible light is used to induce the radical reaction, then the probe and the target protein can be kept in the dark prior to the stimulus being applied. Practically this can be achieved using an opaque reaction vessel. The reaction mixture can then be decanted into a clear vessel to trigger the reaction. Applying a stimulus to induce a radical reaction in the probe-protein complex may comprise exposure to visible light together with the radical initiator eosin (e.g. Eosin Y).

Applying a stimulus may additionally comprise employing one or more radical stabilisers, such as a photo sensitizer. Photosensitizers generally act by absorbing ultraviolet or visible region of electromagnetic radiation and transferring it to adjacent molecules. Suitable photosensitizers include acetophenone compounds, such as 4′-methoxyacetophenone (MAP).

The process may be carried out at standard ambient temperature and pressure (SATP, 25° C. 100 kPa), thereby reducing the risk of degradation of biological samples.

The process may be carried out at 37° C. and ambient pressure for biological relevance.

### Providing the Probe-Protein Complex (e.g. the Probe-Enzyme Complex)

An advantage of the present invention is that labelling takes place when the stimulus is applied. As such, the probe-protein complex can be allowed to equilibrate prior to the stimulus being applied. This can be achieved by a reversible binding reaction between the recognition element of the probe and the recognition site of the target protein.

Without being bound by theory, it is submitted that a binding interaction between the target protein and the probe pre-orders the subsequent radical reaction by aligning the warhead such that the cysteine residue is primed to attack. Conventional probes employ a nucleophilic mechanism which leads to immediate covalent bonding, and a binding equilibrium may not be reached.

Providing the probe-protein complex may comprise combining the target protein and the probe, i.e. incubating the target protein together with the probe for a given period, prior to the stimulus being applied.

The given period (the incubation period) may be 5 minutes or more, 10 minutes or more, 30 minutes or more, 45 minutes or more, 60 minutes or more, or 90 minutes or more; and/or the given period may be 360 minutes or less, 240 minutes or less, 180 minutes or less, 120 minutes or less, 90 minutes or less or 60 minutes or less. In the examples, a binding equilibrium is fully established by 90 minutes.

Target Protein (e.g. Enzyme)

The protein to be labelled comprises a recognition site which has an affinity for the recognition element of the probe. The inventors submit that this affinity provides selectivity for the subsequent labelling.

The examples demonstrate binding to USP5, UCHL3, UCHL1, USP15, USP19, USP7, USP13, FAM188A, OTUB1, USP4, UBP11, OTU1, UBA1, HUWE1, HECTD1, STUB1, UBAS, ARIH1, ARI1 and AR12 (see FIG. 5).

The recognition site may be an active site or an allosteric site.

The target protein may be an enzyme having an active site cysteine, i.e. the cysteine residue may be adjacent the recognition site (active site).

Cysteine (symbol Cys or C) is an amino acid with the chemical formula HO₂CCH(NH₂)CH₂SH. The inventors propose that the thiol group (—SH) forms a thiyl radical (—S′) when the stimulus is applied. The thiyl radical then reacts with the warhead to form a covalent bond. It will be understood that the cysteine residue must be accessible to allow radical reaction with the warhead.

An enzyme having an active site cysteine may be selected from a cysteine protease, a glycosidase, a kinase, a phosphatase, an isomerase, an oxidoreductase, a hydrolase, a thiolase, a sulfurtransferase, or a synthase.

The process of the invention is particularly suitable for the detection of a deubiquitinating enzyme. The enzyme having an active site cysteine may be a deubiquitinating enzyme (DUB), such as OTUB1, OTUB2, or UCHL3, UCHL1 or USP7.

An active site may be described as a region of an enzyme where substrate molecules bind and undergo a chemical reaction. An active site may be a cavity (pocket), e.g. a solvent accessible pocket, as shown in FIG. 11.

The target protein may be an antigen binding polypeptide, such as an antibody or fragment thereof or a T-cell receptor or fragment thereof, and the recognition element may be an antigenic polypeptide recognised by the antigen binding polypeptide.

The target protein may be an antigenic polypeptide and the recognition element may be an antigen binding polypeptide, such as an antibody or fragment thereof or a T-cell receptor or fragment thereof which recognises the antigenic polypeptide.

The invention may be employed to study antigen binding interactions, for example, to assess antibodies or T-cell receptors (non-enzymatic “antigen binding polypeptides”) which bind a known antigenic peptide.

As an example, in this scenario the recognition element of the probe would be the antigenic peptide itself, and a tag may be used if required. Alternatively, no tag would be needed at this stage if known antibodies or T-cell receptors are known which bind the antigenic peptide of interest, as these could be used to pluck out the peptide-binders, analogous to an immunoprecipitation (assuming the known antibodies etc. can still bind the peptide, on different residues).

Vice versa, the recognition element in the probe could be a known T-cell receptor or antibody, or even a fragment such as an ScFv (single chain variable fragment) and the target could be anything it binds, for example antigenic peptides important in clinically relevant immune responses. Again, if other known antibodies or tools are known which bind the T-cell receptor or antibody, these could be used to fish out the binders (if sterically possible), and thus a tag may not always be necessary.

### Probe

The probe can be described as an activity based probe (ABP). The probe comprises a recognition element and a warhead, and optionally a tag.

The tag can be viewed as a detectable label. The tag may comprise a protein tag; an organic molecule tag; and/or a radioisotope tag.

The probe-protein complex may comprise a probe having a tag, i.e. the tag is already present when the stimulus is applied to covalently bond the warhead and the cysteine residue.

A tag may be introduced onto the labelled protein, i.e. after the stimulus is applied.

Protein tags encompass peptide/epitope tags, and include human influenza hemagglutinin (HA); poly histidine; albumin-binding protein; alkaline phosphatase (AP); calmodulin; horseradish peroxidase; LacZ, luciferase; myc epitope; V5; protein C; protein A; protein G; strep-tag; streptavidin; FLAG; glutathione S transferase (GST); green fluorescent protein (GFP); and r-phycoerythrin.

Organic molecule tags include FITC and biotin.

Radioisotope tags include ³H, ¹⁴C or ³⁵S.

In some embodiments, the tag does not comprise N terminal tetramethylrhodamine (TMR). TMR is a bright orange-fluorescent dye. In certain embodiments the tag is not fluorescent.

The recognition element may comprise a protein (or peptide) that is selected based on the target protein (e.g. enzyme) to be labelled. Where the target protein is an enzyme, the recognition element may be based on the natural substrate of the enzyme to be labelled. Hence, when labelling a deubiquitinating enzyme (DUB) the recognition element may be based on ubiquitin. Similarly, when labelling the enzyme UTP-glucose-1-phosphate uridylyltransferase, the recognition element may be based on UTP glucose or an analogue.

The ubiquitin protein has a molecular mass of about 8.6 kDa and consists of 76 amino acids (SEQ ID NO 1, human Ub):

MQIFVKTLTGKTITLEVEPSDTIENVKAKIQDKEGIPPDQQRLIFAGKQLEDGRTLS DYN IQKESTLHLVLRLRGG

The recognition element may comprise an amino acid sequence of ubiquitin or a ubiquitin-like protein. The recognition element may contain one or more amino acid residues in addition to said amino acid sequence, or it may consist essentially of said amino acid sequence. In an embodiment, the recognition element comprises or consists of an amino acid sequence with 90% or more, 93% or more, or 95% or more identity to SEQ ID NO 1.

The amino acid sequence may comprise ubiquitin or any related proteins or fragments thereof that share the same affinity and enzyme activities of ubiquitin, including any affinities and activities within a 10-fold range of that of ubiquitin. The amino acid sequence may comprise from 50 to 100 amino acid residues, e.g. from 50 to 80 amino acid residues, e.g. from 70 to 80 amino acid residues, e.g. 74, 75 or 76 amino acid residues.

The recognition element may comprise or consist of an amino acid sequence selected from ubiquitin (Ub), ubiquitin 1-75 (Ub₇₅), ubiquitin-like 5 (UBLS), ubiquitin-fold modifier 1 (UFM1), autophagy-related protein 12 (ATG12/APG12), autophagy-related protein 8 (ATG8/APG8), ubiquitin-related modifier 1 (URM1), interferon stimulated gene 15 (ISG15), ubiquitin-like protein FAT10, small ubiquitin-like modifier 1/2/3 (SUMO 1/2/3) 3, neural precursor cell expressed, developmental-down-regulated 8 (NEDD8), ubiquitin cross-reactive protein (UCRP), homologous to ubiquitin 1 (HUB1), ribosomal protein S30 fused to a ubiquitin-like protein (Fau) and bacterial ubiquitin-like modifier Pup.

The recognition element may comprise or consist of ubiquitin 1-75 (Ub₇₅). The C-terminal glycine 76 residue that is present in wild-type ubiquitin may be replaced with the warhead (e.g. an alkene moiety).

The warhead reacts with active site cysteine to form a covalent bond via a radical mechanism. As such, the warhead can be viewed as a reactive group which can be attacked by a thiyl radical to form a covalent bond.

The warhead may comprise or consist of an alkene moiety and/or a strained ring system and/or an internal alkyne.

The warhead may comprise cyclopropane or cyclobutane, which may or may not be substituted. Such groups may react with the thiyl radical of the cysteine residue to reduce ring strain.

The warhead may comprise or consist of

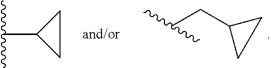

The warhead may comprise or consist of

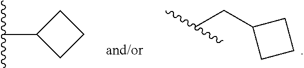 [SMILES: *C1CCC1 ; *CC1CCC1]

The warhead may comprise an alkene in a strained cyclic system, such as norbornene, cyclopropene or cyclobutene, which may or may not be substituted.

The warhead may comprise or consist of norbornene, which may or may not be substituted:

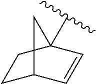 [SMILES: *C12C=CC(CC1)C2]

The warhead may comprise an internal alkyne. A terminal alkyne is not expected to be successful since it could react spontaneously with the cysteine residue via a nucleophilic mechanism.

The warhead may comprise or consist of

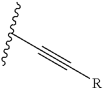

wherein R is selected from an alkyl group, an aryl group, an aralkyl group or a silyl group. In particular, R may be selected from methyl, ethyl, butyl, phenyl, and TMS (tetramethyl silyl).

The warhead may comprise an alkenyl group (an alkene moiety), such as a terminal alkenyl group.

The alkenyl group may have the general structure

R¹C═CR²R³

wherein each of R¹, R² and R³ is independently selected from H, NH₂, an alkyl group, a further alkenyl group, an aryl group and an aralkyl group.

The alkenyl group may have E or Z stereochemistry as determined by the Cahn-Ingold-Prelog priority rules.

The alkyl group may be a straight or branched chain alkyl moiety or a cyclic moiety. The alkyl group may comprise both an acyclic portion and a cyclic portion. The alkyl group may have from 1 to 30 carbon atoms, such as from 1 to 20 carbon atoms, e.g. from 1 to 12 carbon atoms. The alkyl group may have 1, 2, 3, 4, 5 or 6 carbon atoms. Examples of alkyl groups include methyl, ethyl, propyl (n-propyl or isopropyl), butyl (n-butyl, sec-butyl or tert-butyl), pentyl, hexyl, cyclopentyl and cyclohexyl.

The further alkenyl group may be a straight or branched chain alkenyl moiety or a cyclic moiety. The alkenyl group may comprise both an acyclic portion and a cyclic portion. The alkenyl group may have from 2 to 30 carbon atoms and, in addition, at least one carbon-carbon double bond, of either E or Z stereochemistry where applicable. For instance, an alkenyl group may have from 2 to 20 carbon atoms, e.g. from 2 to 10 carbon atoms. In particular, an alkenyl group may have 2, 3, 4, 5 or 6 carbon atoms. Examples of alkenyl groups include ethenyl, 2-propenyl, 1-butenyl, 2-butenyl, 3-butenyl, 1-pentenyl, 2-pentenyl, 3-pentenyl, 1-hexenyl, 2-hexenyl, 3-hexenyl, cyclopentenyl, and cyclohexenyl.

The term “aryl” as used herein refers to an aromatic carbocyclic ring system having from 6 to 30 ring carbon atoms. For instance, an aryl group may have from 6 to 16 ring carbon atoms, e.g. from 6 to 10 ring carbon atoms. An aryl group may be a monocyclic aromatic ring system or a polycyclic ring system having two or more rings, at least one of which is aromatic. Phenyl is an example of an aryl group.

The term “aralkyl” as used herein refers to an alkyl group substituted with an aryl group, wherein the alkyl and aryl groups are as defined herein. An example of an aralkyl group is benzyl.

The alkyl/alkenyl/aryl/aralkyl group may consist exclusively of hydrogen and carbon atoms group. Alternatively, the alkyl group/alkenyl/aryl/aralkyl group may be substituted, optionally with OH or NH₂.

Electron withdrawing groups (e.g. C═O, NO₂, CN etc) should be avoided, to encourage radical reaction rather than a nucleophilic reaction.

In one embodiment R¹ may be H or an alkyl group, e.g. H or alkyl having from 1 to 30 atoms (e.g. 1 to 20, 1 to 12, or 1 to 6 carbon atoms).

In one embodiment R² and/or R³ may be H or an alkyl group, e.g. H or alkyl having from 1 to 30 atoms (e.g. 1 to 20, 1 to 12, or 1 to 6 carbon atoms).

In one embodiment R² and/or R³ may be an alkyl, alkenyl, aryl or aralkyl group, having from 1 to 30 atoms (e.g. 1 to 20, 1 to 12, or 1 to 6 carbon atoms).

R¹ may be H; and/or R² may be H; and/or R³ may be H.

R¹ may form an alkenyl ring together with R² or R³, e.g. R¹ together with R² or R³ may form a cyclopentene, a cyclohexene or norbornene.

R² and R³ may together form an alkyl ring. e.g. R² and R³ may together form a cyclopentane or a cyclohexane.

It will be understood that the probe is different from those used in the conventional probes which form a covalently bond immediately via a nucleophilic mechanism.

The warhead may not comprise C≡O, C═N, C═N, C≡N, N═O and/or S═O bonds. The warhead may not comprise a halide (—F, —Cl, —Br or —I). These are electron withdrawing groups (EWGs) and may function as an electrophilic trap, thereby competing with the radical reaction.

The warhead may comprise the general structure (I):

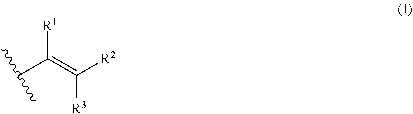

wherein each of R¹, R² and R³ is independently selected from H, NH₂, an alkyl group, a further alkenyl group, an aryl group and an aralkyl group, as defined above.

In particular, the warhead may comprise the general structure (II) or (III)

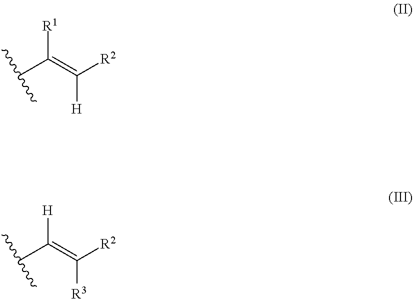

wherein each of R¹, R² and R³ is independently selected from H, NH₂, an alkyl group, a further alkenyl group, an aryl group and an aralkyl group, as defined above. The examples demonstrate successful labelling using a probe where each of R¹, R² and R³ is H (probe 1), i.e. where the warhead comprises general structure (IV):

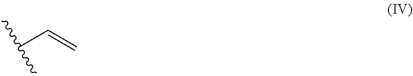 [SMILES: *C=C]

Labelling was also achieved where each of R¹ and R² is H and R³ is phenyl (probe 2), i.e. where the warhead comprises general structure (V)

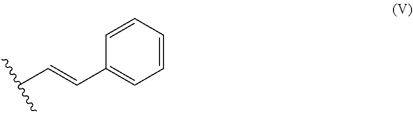 [SMILES: */C=C/c1ccccc1]

Labelling was also achieved where each of R¹ and R² is H and R³ is methyl (data not shown), i.e. where the warhead comprises general structure (VI)

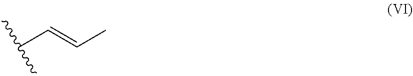 [SMILES: */C=C/C]

The wavy line represents the remainder of the probe.

Where the recognition element has an amino acid sequence, the warhead may be attached at the carboxy terminus of said amino acid sequence as in general structure (VII):

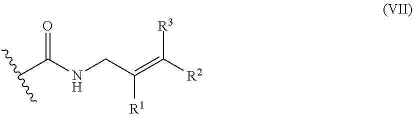

The skilled person will appreciate that features of any one embodiment and/or aspect of the invention may be applied to all other embodiments and/or aspects of the invention.

According to a second aspect of the invention there is provided a probe-protein complex for use in the process of the first aspect of the invention,

the probe-protein complex comprising a probe and a target protein;
the probe comprising a recognition element and a warhead; and
the target protein comprising a cysteine residue and a recognition site;
wherein the recognition element is reversibly bound to the recognition site.

It will be understood that the warhead and the cysteine residue are capable of a radical reaction to covalently bond the warhead to the cysteine residue.

The probe may additionally comprise a tag. The target protein, the tag, the recognition element and the warhead are described above.

In some embodiments the tag is a protein tag or a radioisotope tag.

According to a third aspect of the invention, there is provided a probe for use in the process of the first aspect of the invention and/or the complex of the second aspect of the invention, the probe comprising a tag, a recognition element and a warhead, wherein the warhead is capable of a radical reaction with a cysteine residue to covalently bond the warhead to the cysteine residue.

The tag, the recognition element and the warhead are described above.

In some embodiments the tag is a protein tag or a radioisotope tag.

In some embodiments, the probe comprises a warhead having the general structure I wherein at least one of R¹, R² and R³ is independently selected from NH₂, an alkyl group, a further alkenyl group, an aryl group and an aralkyl group.

In some embodiments, the warhead comprises an alkenyl group having the general structure I, wherein each of R¹, R² and R³ is not H.

In some embodiments the warhead comprises a strained ring system and/or an internal alkyne.

According to a fourth aspect of the invention, there is provided a labelled protein (e.g. a labelled enzyme) comprising

a target protein covalently bonded to a probe via a thiol linkage,
the probe comprising a tag and a recognition element.

The target protein, the tag, and the recognition element are as described above.

The thiol linkage may have the structure VIII

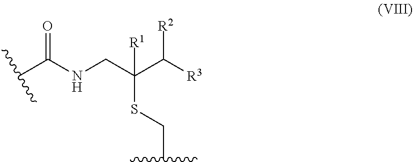

wherein each of R¹, R² and R³ is independently selected from H, an alkyl group, an alkenyl group, an aryl group or an aralkyl group.

### BRIEF DESCRIPTION OF THE FIGURES

The present invention will be further described in more detail, by way of example only, with reference to the following figures in which:

FIG. 1—shows the MS analysis of intact probe 1 using MALDI, calculated m/z 10062 (M+H)⁺ and 5032 (M+2H)²⁺, found m/z 10061 (M+H)⁺ and 5034 (M+2H)²⁺. (B) LC-MS/MS identification of Ub₇₅ C-terminal peptide of probe 1. (C) Purified probe 1 was resolved using 12% SDS-PAGE and visualised using silver staining.

FIG. 2—shows the labelling of recombinant OTUB1 (2 μg) with probe 1 (4 μg). Visualised using (A) Anti-HA western blot and (B) Silver stain. Recombinant OTUB1 (2 μg) was denatured using heat or SDS prior to incubation with the probe 1 (4 μg) in lanes 3 and 4. Probe 1 was pre-incubated for 90 min prior to addition of initiators and exposure to UV light (365 nm) for 5 min.

FIG. 3—shows A) HEK 293T cell lysate (50 μg) was incubated with probe 1 (1 μg) for 60 min before exposure to UV light (365 nm) for a range of times. Controls included using NEM (10 mM), the Ub₇₅CH₂CH₂Br probe (1 μg) and a labelling excluding initiators. (B) HEK 293T cell lysate (50 μg) was pre-incubated for varying periods of time with probe 1 (1 μg) before exposure to UV light (365 nm) for 2 min. Controls included no degassing and an inactive probe (1 μg) incubated for 45 min.

FIG. 4—shows that the probe binds DUBs and can compete with a known DUB inhibitor, whereas known probes cannot. (A) HEK 293T cell lysate (50 μg) was pre-incubated for 90 min with increasing concentrations of PR-619 before being labelled with the probe 1 (1 μg). (B) HEK 293T cell lysate (50 μg) was pre-incubated with probe 1 (1 μg) for 90 min before PR-619 was added at increasing concentrations. (C) HEK 293T cell lysate (50 μg) was incubated with Ub₇₅CH₂CH₂Br and PR 619 (100 μM) was added to one sample. All samples were exposed to UV light (365 nm) for 2 min.

FIG. 5—is a Heatmap showing the enrichment of DUBs and ubiquitin conjugation machinery after immunoprecipitation with anti-HA coupled agarose beads using probe 1, probe 2 and lysate alone. Intensities calculated using label-free quantification following LC-MS/MS analysis. The replicate that had the highest intensity for each individual enzyme was taken as 100% and the values of other replicates for the enzyme are expressed as a percentage of this value.

FIG. 6—shows the labelling of recombinant DUB OTUB1 (3 μg) with probe 1 (6 μg) was visualised by (A) anti-His western blotting and (B) silver staining. Following separation by SDS-PAGE. Indicated bands on the silver stain were digested and analysed by captive spray ionisation mass spectrometry. Protein coverage refers to the % of the proteins sequence identified.

FIG. 7—demonstrates the K_{d} of probe 1 with OTUB1 (A) Recombinant DUB OTUB1 (5 μg) was labelled with increasing concentrations of probe 1. Labelling was visualised by Coomassie blue staining following separation by SDS-PAGE. (B) Intensity of the labelled band was analysed on Image Quant and plotted against the concentration of probe.

FIG. 8—shows the optimisation of conditions of labelling of enzymes with the probe. The probe was incubated with HEK293T lysate for 90 min before the radical initiator 2,2-dimethoxy-2-phenylacetophenone (DPAP) was added at varying concentrations along with the radical stabiliser methoxyacetophenone (MAP). Samples were degassed and exposed to UV light (365 nm) for 2 min. (B) Time under UV was investigated using DPAP and MAP (0.25 μM) to improve compatibility with LC-MS/MS. The initiators were added to the samples after a 90 min incubation and exposed to UV light for the time indicated.

FIG. 9—shows that the integrity of the probe and cell lysate is maintained under different conditions. HEK 293T cell lysate (50 μg) was pre-incubated with probe 1 (1 μg) for 90 min before PR 619 was added at increasing concentrations. Samples were separated on 12% SDS-PAGE and visualised by silver staining.

FIG. 10—shows that WT-Ub competes with the probe to bind deubiquitinating enzymes before activation with UV light, disrupting the equilibrium when added in excess. The probe (1 μM) was incubated with HEK 293T cell lysate (50 μg) for 90 min. WT ubiquitin was added at increasing concentrations and incubated for 30 min before addition of radical initiators and exposure to UV light (365 nm).

FIG. 11—demonstrates the proposed mechanism of covalent bond formation of probes to DUBs via a thiol-ene reaction following the establishment of a binding equilibrium.

FIG. 12—visible light-mediated activity-based protein profiling of DUBs using the photocatalyst Eosin Y 2.

FIG. 13—labelling of OTUB1 (2 μg) with probe 1 (3 μg) visualised by anti-HA western blot. Samples were preincubated for 90 min at 37° C. before the addition of Eosin Y. Samples were incubated for a further 10 min at 37° C. in the dark (lane 3) in ambient light (lane 4) or exposed to a 10 W white light lamp from 10 cm (lane 5).

FIG. 14—concentration dependence of Eosin Y for an OTUB1 (2 μg) labelling with probe 1 (3 μg) visualised by anti-HA western blot. Samples were preincubated for 90 min at 37° C. before the addition of Eosin Y at the indicated concentration. Samples were incubated for a further 30 min at 37° C. in ambient light.

FIG. 15—time dependence of ambient light exposure for an Eosin Y mediated labelling of OTUB1 (2 μg) with probe 1 (3 μg) visualised by anti-HA western blot. Samples were preincubated for 90 min at 37° C. before the addition of Eosin Y (5 μM). Samples were incubated for a further 30 min at 37° C. in ambient light.

FIG. 16—the effect of degassing on OTUB1 (2 μg) labelling with probe 1 (3 μg) visualised by anti-HA western blot. Samples were preincubated for 90 min at 37° C. Lane 4, 6 and 8 were degassed for 2 min using N₂ before Eosin Y was added to all samples. A further incubation was carried out for 10 min at 37° C. while exposed to a 10 W white light lamp from 10 cm.

FIGS. 17—10 W white light lamp distance variation for Eosin Y mediated OTUB1 (2 μg) labelling with probe 1 (3 μg) visualised by anti-HA western blot. Samples were preincubated for 90 min at 37° C. before the addition of Eosin Y at the indicated concentration. Samples were incubated for a further 30 min at 37° C. in ambient light.

FIG. 18—comparative analysis of labelling using Eosin Y as the initiator relative to DPAP and MAP for recombinant enzymes OTUB1 (2 μg) and UCHL1 (2 μg), lanes 2-4 and 5-7, with probe 1 (3 μg) visualised by anti-HA western blot. Samples were preincubated for 90 min at 37° C. Lanes 3 and 6 were degassed for 2 min using N₂ before the addition of DPAP and MAP or Eosin Y to the indicated samples. A further incubation was carried out for 10 min at 37° C. while lanes 3 and 6 were exposed to UV light (365 nm) and lanes 4, and 7 were exposed to a 10 W white light lamp from 50 cm.

### DETAILED DESCRIPTION

### Example 1

Firstly, a probe was generated using semi-synthetic techniques, employing an activated thioester intermediate as previously described (A. Borodovsky, B. M. Kessler, R. Casagrande, H. S. Overkleeft, K. D. Wilkinson and H. L. Ploegh, EMBO J, 2001, 20, 5187-5196). Its structure is comprised of a human influenza hemagglutinin (HA) tag and ubiquitin 1-75 (Ub₇₅). The C-terminal glycine 76 residue that is present in wild-type ubiquitin is replaced with an alkene moiety, initial studies focused on a terminal alkene (FIG. 11, R═H).

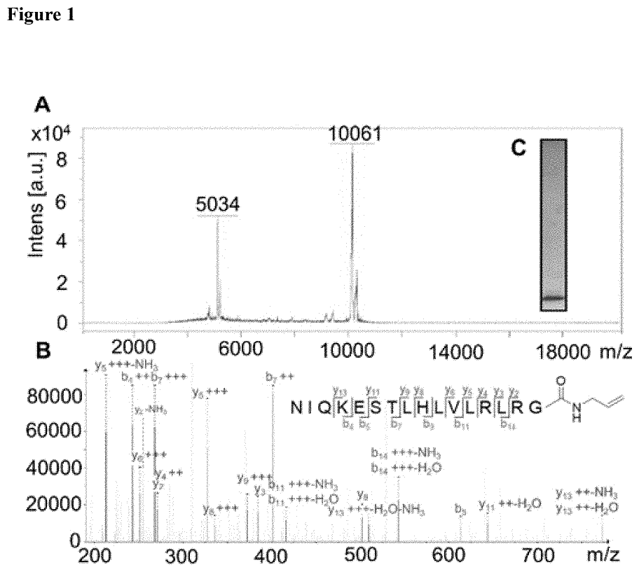

The intact probe was characterised using MALDI mass spectrometry and silver staining, with both showing a single protein of the expected molecular weight (FIGS. 1A and 1C). LC MS/MS located the allyl amide functionality to the C terminus of the protein, confirming the structure of the probe (FIG. 1B).

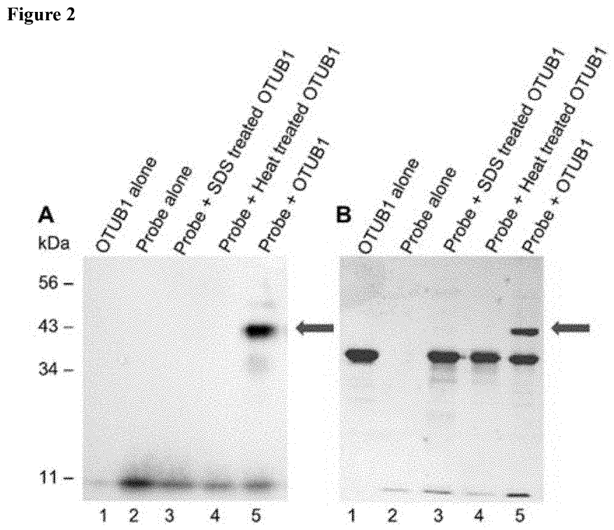

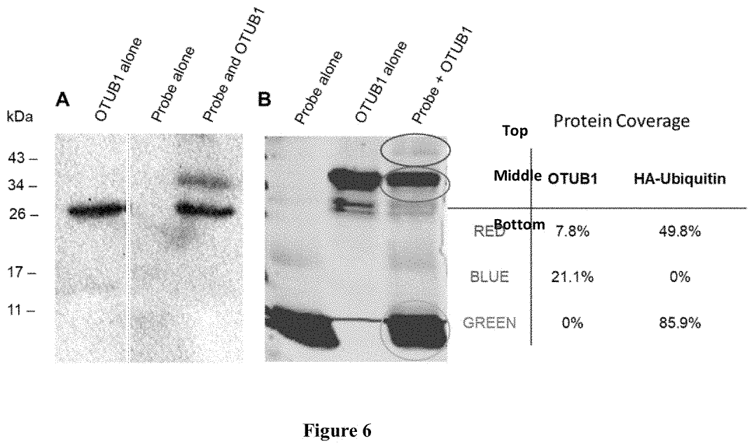

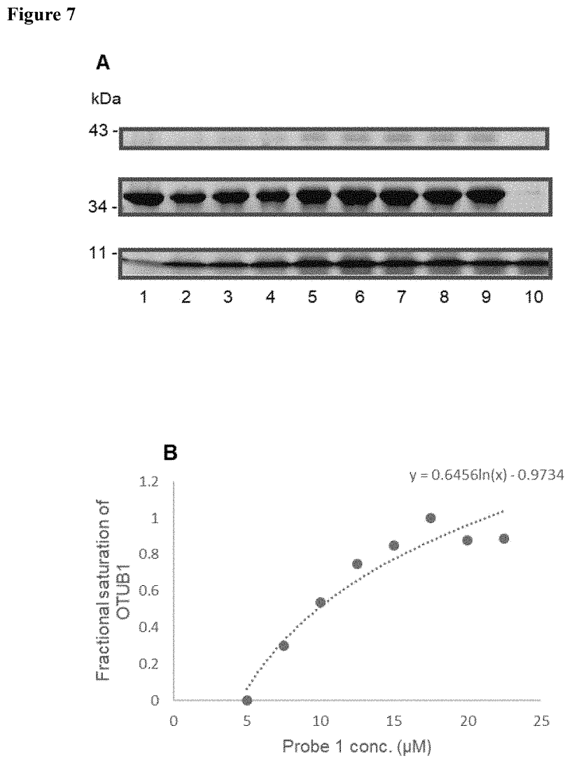

Validation of whether probe 1 could react with DUBs was then undertaken, using His-tagged, recombinant DUB OTUB1 (FIG. 2). Following a 60 minute pre-incubation at 37° C., the radical initiator 2,2-Dimethoxy-2-phenylacetophenone (DPAP) was added along with radical stabiliser 4′-Methoyacetophenone (MAP). Samples were then degassed with N₂ for 2 min prior to exposure to UV light (365 nm). UV light was applied in a photoreactor with six top lamps and eight side lamps. The power rating provided by the manufacturer (Luzchem) is 110 VAC/220 VAC, 50/60 Hz cycle, 3 amps. The reaction was analysed by anti-His western blots (FIG. 2A-2B and FIG. 6). After labelling, a new band corresponding to the expected molecular weight of the probe-enzyme adduct is observed in lane 5 of both gels, as indicated by the arrow. When OTUB1 was denatured using heat or SDS, no new band is visible. This validates the need for a specific binding interaction between the probe and the active form of an enzyme for the reaction to occur. In-gel digestion of the new band confirmed the presence of both the probe and OTUB1 (FIG. 6). Further to this, elucidation the K_{d} of the probe was demonstrated using OTUB1 as a model system. A K_{d} of 7.8 μM was derived and applied in combination with reversible inhibitors, this system could be a simple technique to evaluate their potency (FIG. 7).

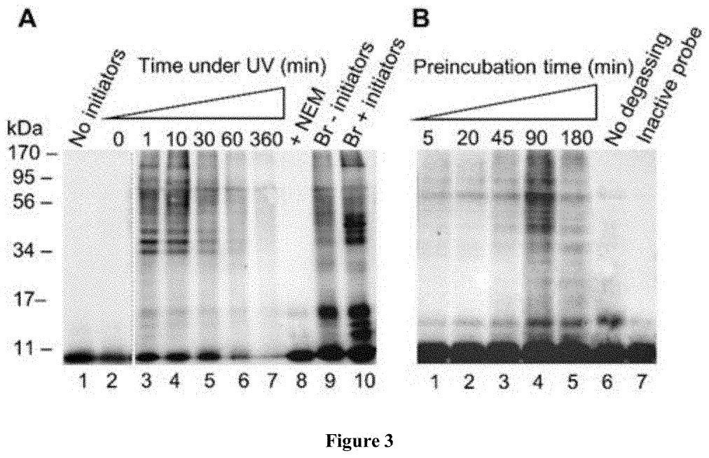

After confirming reactivity with a recombinant DUB, probe 1 was tested in HEK293T cell lysate using conditions analogous to those described for the recombinant enzyme labelling (FIG. 3). The absence of detectable labelling in lane 1 confirms the requirement of radical initiation for covalent labelling (FIG. 3A). Exposure time to UV light was optimised, with conditions tested from 0 to 360 minutes (FIG. 3A, lanes 2-7). No labelling is observed at the 0 minute time point (FIG. 3A, lane 2). There is no discernible difference between the 1 minute and 10 minute timepoints (FIG. 3A, lanes 3 and 4). One minute of UV light exposure is sufficient for complete labelling under these conditions. This suggests the binding interaction between the enzyme and probe 1 has pre-ordered the reaction by aligning the C-terminal alkene such that the active site cysteine is primed to attack. Prolonged exposure to UV light showed degradation of the sample at longer timepoints (FIG. 3A, lanes 5-7). Two further control experiments were performed to validate the need for free thiols and to compare to an existing probe. Comparison between lanes 8 and 4 reveals that the addition of thiol alkylator N-ethylmaleimide (NEM) inhibited labelling (FIG. 3A). Covalent bond formation therefore requires free cysteine residues. A labelling reaction with the known HA-Ub₇₅CH₂CH₂Br probe was subjected to the same conditions as the thiol-ene labelling to confirm there was no degradation of the lysate under these conditions (FIG. 3A, lanes 9 and 10).

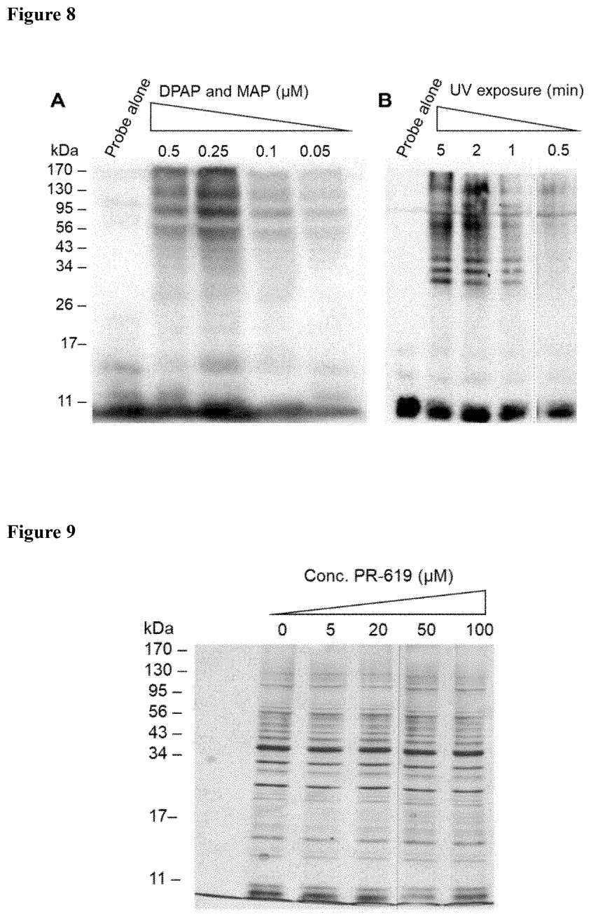

It was next examined how the pre-incubation period affected the labelling pattern (FIG. 3B, lanes 1-5). It was reasoned that prior to activation, a binding equilibrium must be established between the probe and target enzymes to yield the rapid labelling observed. The conditions of the labelling were altered slightly from the recombinant enzyme labelling with a shortened UV light exposure time of 2 min and variations in the pre-incubation time. After a 5 minute incubation there was little visible labelling. Increasing timepoints from 20 to 90 minutes correlated to an increase in the intensity of the labelling pattern. A drop-off is observed by the 180 minute timepoint, indicating that the binding equilibrium is fully established by 90 minutes. This drop-off can likely be attributed to a reduction in DUB activity after an extended incubation time in cell lysate. These results were consistent with the hypothesis that a pre-organised reaction is occurring following a specific binding interaction. Two further experiments were carried out to confirm the thiol-ene reaction is responsible for the covalent labelling. Samples were degassed prior to exposure to UV light to reduce the influence of reactive oxygen species which have been demonstrated to be inhibitory to certain DUBs.^{[30]} Omitting this step reduced labelling (FIG. 3B, lane 6 vs lane 5). The absence of labelling when HA-Ub₇₅ is used confirms that the alkene moiety is necessary for covalent bond formation (FIG. 3B, lane 7). These results, in combination with the findings that the reaction does not proceed when cysteine residues are alkylated and when initiators are absent, provide compelling evidence that the thiol-ene reaction is responsible for the observed labelling. Optimisation was also carried out for initiator concentration along with more refined optimisation of time under UV (FIG. 8).

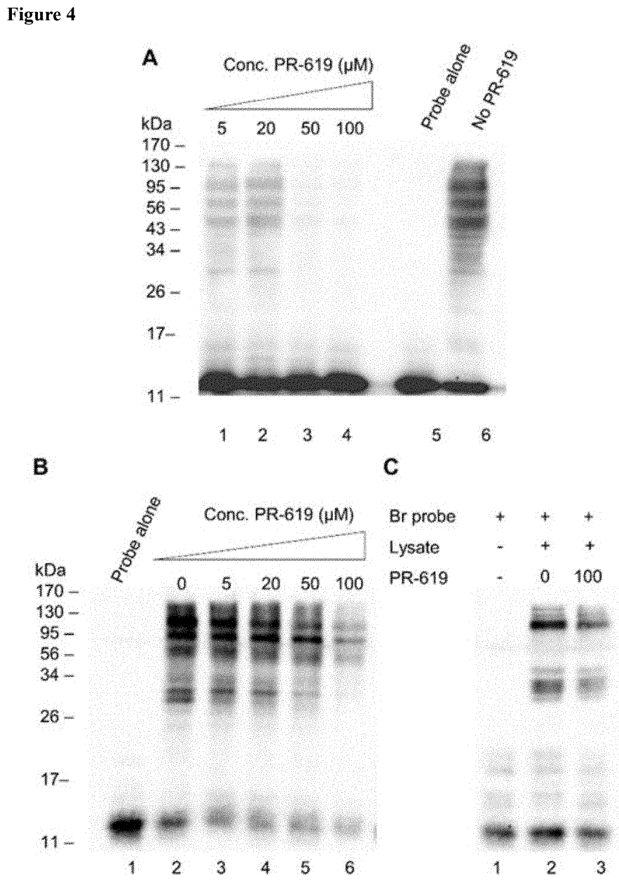

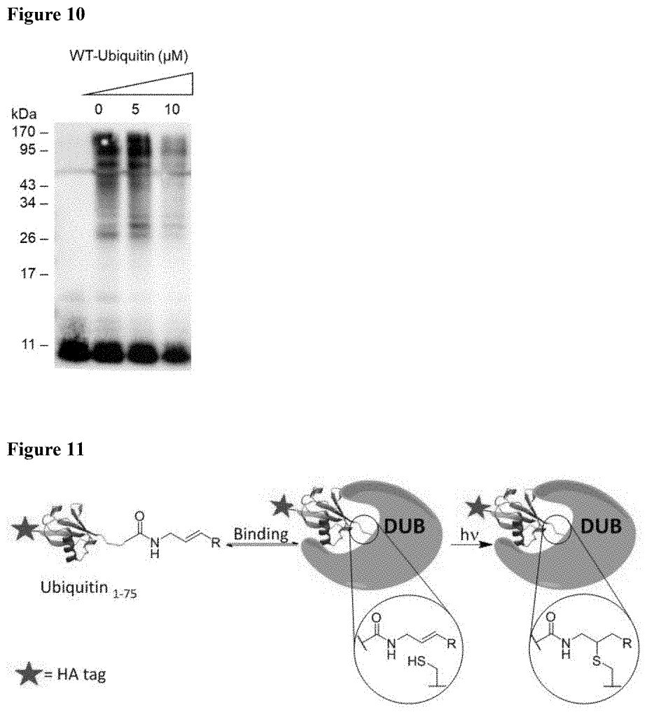

It was next set out to test the modulation of labelling using the known pan DUB inhibitor, PR-619 (X. Tian, N. S. Isamiddinova, R. J. Peroutka, S. J. Goldenberg, M. R. Mattern, B. Nicholson and C. Leach, Assay Drug Dev Technol, 2011, 9, 165-173). PR-619 was incubated with cell lysate for 30 min prior to the addition of probe 1 and the labelling was then carried out under optimised conditions with a 90 min pre-incubation time. A clear concentration-dependent reduction in labelling intensity was observed when the lysate was pre-incubated with PR-619 (FIG. 4A, lanes 1-6). This implies specificity of probe 1 towards DUBs. To demonstrate how the thiol-ene mediated labelling mechanism can be used advantageously in inhibitor studies, an additional experiment was performed whereby the binding equilibrium was established between the probe 1 and DUBs in lysate during a 90 min incubation. PR-619 was titrated into the mixture at increasing concentrations and incubated for a further 30 min before the addition of initiators and exposure to UV light (FIG. 4B). A parallel experiment was set up with the Ub₇₅CH₂CH₂Br probe for comparison (FIG. 4C). A similar trend is observed to the previous experiment where PR-619 abrogates the labelling in a concentration dependent manner when using the probe 1 (FIG. 4B). When the conditions of lanes 2 and 6 (0 μM and 100 μM PR-619 respectively), are replicated with the Ub₇₅CH₂CH₂Br probe, only minor difference is observed. (FIG. 4C, lanes 2 and 3). To check the integrity of the probe and cell lysate under these conditions a silver stain of the samples was also performed (FIG. 9). This demonstrates how DUB inhibitors can disrupt the binding equilibrium, thus allowing the study of these inhibitors in a novel way with greater control. Similarly, analogous effects using wild-type ubiquitin in place of PR-619 were observed, illustrating how probe 1 can be utilised study reversible binding events (FIG. 10).

In order to extend the scope of thiol-ene mediated DUB capture and explore its structural requirements, a phenyl-substituted alkene probe 2 (Scheme 1, R=Ph) was synthesised to examine if a more substituted alkene would alter reactivity and selectivity. Probe 2 was tested in HEK 293T lysate alongside the terminal alkene probe 1 which showed a different, generally reduced, labelling pattern by western blot. Despite the more modest labelling observed with phenyl-substituted probe 2, it was taken forward for further comparative analysis with terminal alkene probe 1.

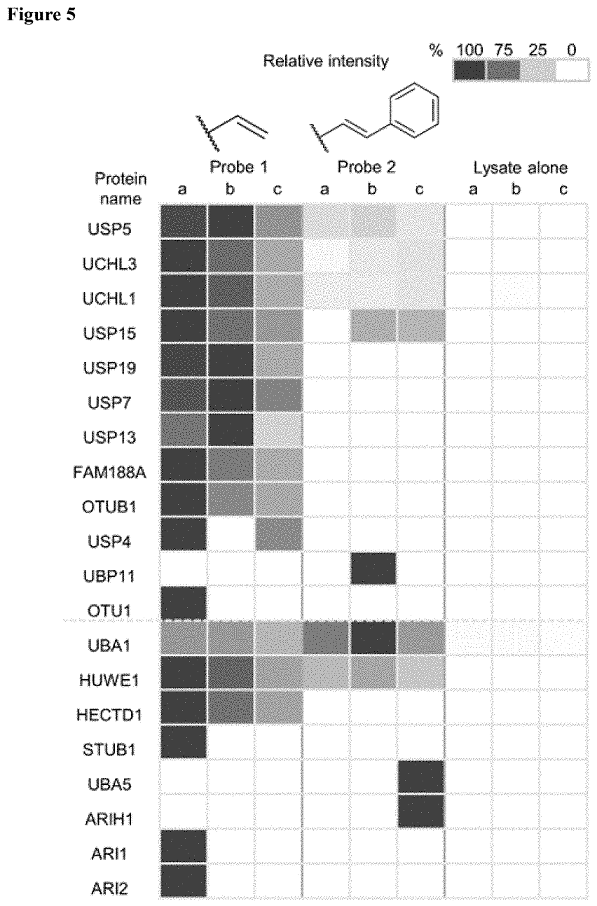

To unambiguously characterise the specificity and reactivity of the probes in complex cellular environments, an immunoprecipitation (IP) was performed on material captured by a thiol-ene labelling. Tagged proteins were enriched and analysed by anti-HA western blot (SI) and LC-MS/MS following tryptic digest (FIG. 5). Both the terminal alkene probe 1 and the phenyl-substituted alkene probe 2 were used along with a probe-free negative control condition, each carried out in triplicate. The inputs and eluates for this experiment were visualised using anti-HA western blot (not shown).

Protein intensities were calculated using label-free quantification with the replicate that had the highest intensity for each individual enzyme being taken as 100%. The values of other replicates are expressed as a percentage of this. Twelve DUBs were identified from the IP as well as eight components of the ubiquitin conjugation machinery. Enrichment of all but one of the identified DUBs was observed in samples treated with the terminal alkene probe 1 (FIG. 5). Enrichment of certain DUBs was also observed for the phenyl-substituted probe 2, although this is less pronounced. It appears the terminal alkene probe 1 yields more efficient capture in this case. Additionally, enrichment of ubiquitin conjugation machinery was also observed in both cases. The probe design is therefore also applicable to labelling the less nucleophilic active site cysteines of these enzymes.

### Conclusion

In conclusion, a novel activity-based monoubiquitin probe has been developed that is completely inactive under ambient conditions and can be selectively activated to label DUBs and enzymes of the ubiquitin conjugation machinery through the active site cysteine. The probe's reactivity against recombinant, purified DUB OTUB1 and HEK 293T lysate has been demonstrated. Experiments using heat and SDS treated recombinant enzyme showed the probe is selective for the active form of the DUB, requiring a specific binding interaction. The ability of the probe to study the effects of inhibitors displacing bound probe has been demonstrated, potentially accelerating development of novel inhibitors. Moreover, it is demonstrated that selectivity can be influenced by the addition of groups proximal to the alkene moiety opening the potential to develop probes more specific to particular DUB classes. Finally, for the first time, the successful application of thiol-ene chemistry in activity-based protein profiling has been demonstrated.

The short activation period of the probe following specific binding provides a snapshot of the DUBs bound at that time. The consequence of capturing the equilibrium in such a way provides an opportunity to study binding affinity and inhibitor potency in new ways for both reversible and irreversible binding interactions. Unlike existing photocrosslinking probes, this technique offers a residue specific, mechanism-based method to provide a time-resolved readout of enzyme activity rather than protein binding. With careful optimisation this approach could be explored for its applicability to intact cells. This radical labelling approach is broadly applicable and provides further opportunities in chemical proteomics beyond the study of reactivity and selectivity in the ubiquitin system as any enzyme bearing an active site cysteine could be targeted in this way. The alkene moiety is chemically inert and sterically undemanding to introduce to other biological entities, such as carbohydrates, DNA or inhibitor scaffolds to access a wide variety of enzyme classes.

### Example 2

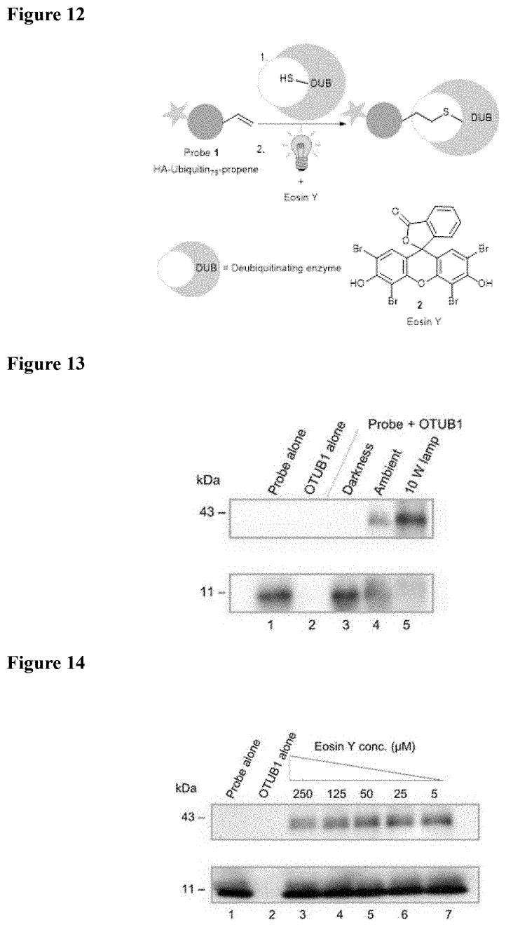

Example 1 (above) presents a method to profile DUB activity in cell lysate using the radical-mediated thiol-ene coupling reaction. A probe consisting of a Hemagglutinin (HA) tag and a Ubiquitin75 recognition element functionalised with a C-terminal alkene moiety, probe 1, was coupled to the active-site cysteine of DUBs. The radical initiator 2,2-dimethoxy-2-phenylacetophenone (DPAP) was used for the coupling along with radical stabiliser 4′-methoxyacetophenone (MAP). The required UV light exposure time and degassing step reduce the biocompatibility of this methodology. In this example we aimed to improve the biocompatibility of the coupling by using the organic photocatalyst Eosin Y 2 to promote a visible light-mediated thiol-ene coupling between the probe 1 and the active-site cysteine of DUBs (FIG. 12).

We hypothesised that removing the requirement for UV light and the degassing step used in the previous methodology would not only improve the biocompatibility of the reaction but also provide a simplified labelling strategy, facilitating its transfer to more complex biological systems.

Firstly, the reactivity of the probe with the recombinant DUB OTUB1 was assessed using Eosin Y as the sole photoinitiator (FIG. 13). Triethanolamine, or similar coinitiators, could be used in combination with Eosin Y. The reaction was initiated using ambient light or a 10 W white light bulb. As a control, an equivalent sample was kept in darkness for the duration of the experiment. All samples containing the probe 1 and OTUB1 were preincubated for 90 min before the addition of Eosin Y to allow for a protein-protein binding equilibrium to be established as previously optimised.

In both samples exposed to visible light a new band was formed around 42 kDa, the molecular weight of a covalent adduct between the probe 1 and OTUB1 (FIG. 13). The same band was not seen in the non-irradiated sample, indicating the adducts seen in lanes 4 and 5 were the result of a light-dependent radical reaction. Protein degradation, represented by loss of signal and streaking of the sample, was observed for the light exposed reactions (FIG. 13, Lanes 4 and 5). To overcome this, lower concentrations of Eosin Y were investigated using ambient light to initiate the coupling (FIG. 14).

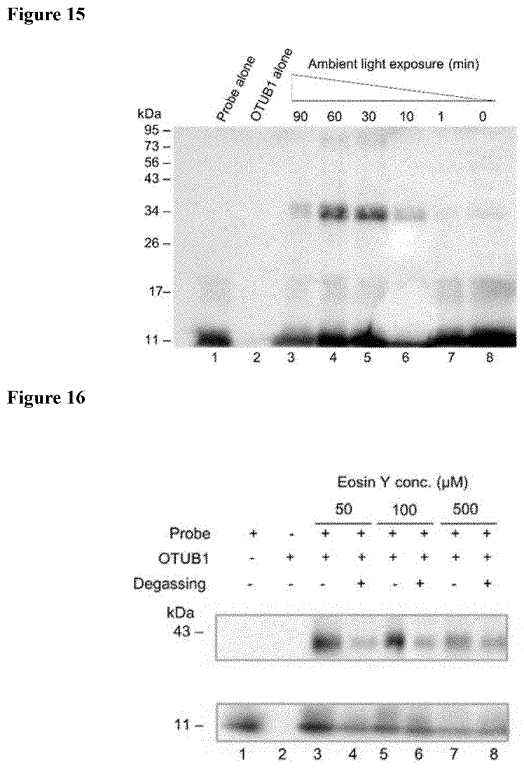

A new band around 43 kDa was once again observed in all samples containing the probe 1, OTUB1 and Eosin Y that were exposed to visible light (FIG. 14, lanes 3-7). Lower concentrations of Eosin Y afforded more efficient coupling when analysed by anti-HA western blot, consistent with the hypothesis that higher Eosin Y concentrations were causing protein degradation. The retention of strong labelling at significantly lower concentrations of Eosin Y was a promising improvement to the biocompatibility of the reaction. Significantly, these couplings were all carried out without a degassing step, therefore in the presence of O₂. Additional optimisation was carried out by varying the incubation time following the addition of Eosin Y. Labelling intensity increased with time until a peak in OTUB1 capture at 30 mins (FIG. 15).

Lower Eosin Y concentrations were also examined using a 10 W source of white light. Additionally, the effect of degassing was also assessed during this experiment (FIG. 16).

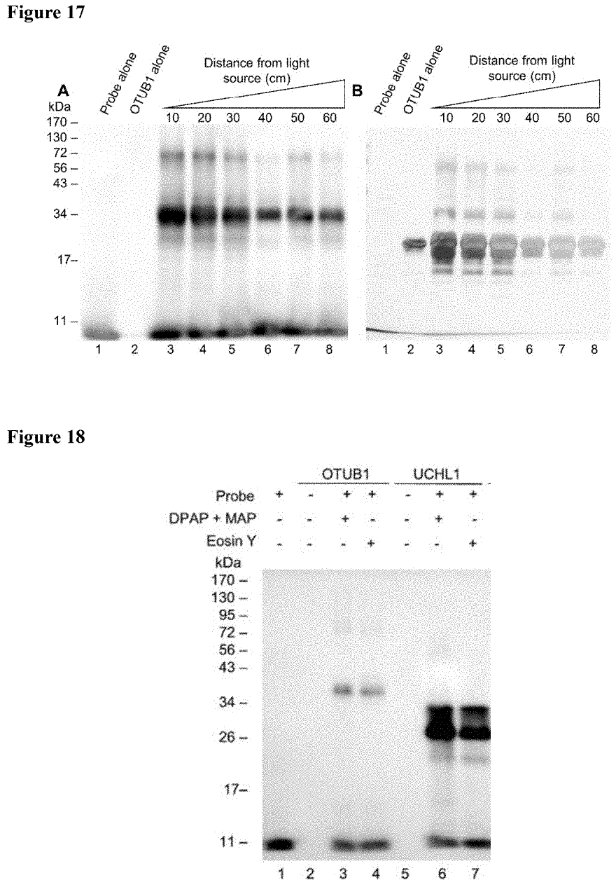

As observed in the ambient light experiments, higher Eosin Y concentrations caused protein damage, with lower concentrations demonstrating increased protein capture. Interestingly, lower protein capture was observed when a degassing step was included. This indicated a potential role for reactive oxygen species (ROS) in the reaction. All further experiments were therefore carried out without a degassing step. Further recombinant enzyme labelling experiments were performed to optimise the distance of the 10 W lamp from the samples (FIG. 17). In these experiments an additional band was evident at approximately 75 kDa. This MW is consistent with the expected MW of an OTUB1 dimer bound to probe 1. This result indicated some off-target crosslinking was occurring. This observation was more pronounced at closer distances, suggesting its formation was radical dependent.

Once optimised, a comparative labelling was carried out between these optimised Eosin Y labelling conditions versus the conditions optimised for DPAP and MAP labelling (FIG. 18). At this point to check the generality of the method, a further DUB, UCHL1 was tested alongside OTUB1. In experiments with both recombinant enzymes, probe capture was comparable using Eosin Y relative to DPAP and MAP.

### Conclusions

In conclusion, Eosin Y was investigated as a photocatalyst for the ABP of DUBs via a visible light-mediated thiol-ene coupling. Promising initial results were obtained using a system consisting of probe 1 and the recombinant DUB OTUB1. Using this system an enzyme-substrate binding interaction must occur before coupling, requiring only approximately a four-fold molar excess of probe relative to enzyme to achieve coupling. In this system Eosin Y appears to be a viable alternative to the DPAP and MAP catalysed coupling. Labelling was achieved at micromolar concentrations of the initiator without the need for UV light or a degassing step, improving the biocompatibility of the reaction. These couplings are, to our knowledge, the first example of a visible light-mediated thiol-ene coupling between two proteins.

In summary, visible light induced thiol-ene coupling using Eosin Y demonstrated efficiency of this coupling using low concentrations of Eosin Y and in the presence of oxygen mean it may provide significant opportunities in the context of protein-protein conjugations.

### Materials and Methods

SDS-PAGE: Proteins were separated on a 12% acrylamide gel (resolving gel: 1.3 mL 1.5 M Tris pH 6.8, 1.5 mL 40% acrylamide/Bis-acrylamide (29:1), 2 mL dH₂O, 50 μL 10% SDS (sodium dodecyl sulfate), 50 μL 10% ammonium persulfate (APS), 5 μL Tetramethylethylenediamine (TEMED); stacking gel: 630 μL 0.5 M Tris pH 6.8, 300 μL acrylamide, 1.3 mL dH₂O, 25 μL 10% SDS, 25 μL 10% APS, 2.5 μL TEMED). Samples were prepared for separation by adding 2× reducing sample buffer (0.2 M Tris pH 6.8, 30% glycerol 0.4% β-mercaptoethanol, 9% SDS, bromophenol blue) followed by heating at 95° C. for 5 min. The proteins were loaded along with Fisher's EZ-Run™ Pre-Stained Rec Protein Ladder. Separation was achieved at 150 V for 1-2 h and visualised either by western blotting or silver staining. All gels were imaged using Chemidoc XRS+(Biorad, California USA) and Typhoon FLA9500 (GE Healthcare, Illinois USA).

Silver staining: Gels were treated with fixative (40% EtOH, 10% AcOH) at rt for 1 h or at 4° C. for 16 h. Gels were washed in 20% EtOH (2×10 min), then in dH₂O (2×10 min). Gels were sensitised in aq. Na₂S₂O₃ (0.02%) for 45 s and then immediately washed with dH₂O (2×1 min). The gel was incubated in a solution of AgNO₃ (12 mM) with formaldehyde (0.02%) at 4° C. for a minimum of 20 min and up to 2 h. Following this, the gel was washed in dH₂O (2×30 s) and transferred to developer solution (3% K₂CO₃, 0.05% formaldehyde). Development was stopped using 5% AcOH.

Western Blotting: Proteins were transferred onto nitrocellulose membranes (GE Healthcare, Illinois USA) in blotting transfer buffer (25 mM Tris, 190 mM glycine, 20% MeOH) overnight at 15 V and 4° C. The membrane was incubated in blocking solution (5% skimmed milk powder in PBST: 8 mM Na₂HPO₄, 150 mM NaCl, 2 mM KH₂PO₄, 3 mM KCl, 0.1% Tween 20, pH 7.4) for 1 h at rt or 16 h at 4° C. prior to immunoblotting. The primary mouse monoclonal anti-HA antibody (Biolegend, California USA) was diluted 1:2000 in blocking buffer and incubated with the membrane for 1 h at rt with gentle shaking. The membrane was washed with PBST (2×5 min) and PBS (2×5 min). The secondary antibody (Jackson ImmunoResearch, Cambridgeshire UK) was diluted in blocking buffer 1:4000, added to the membrane and incubated for 1 h at rt with gentle shaking. The membrane was washed with PBST (3×5 min), PBS (2×5 min) and dH₂O (1×5 min). Pierce ECL western blotting substrate (Thermofisher, Massachusetts USA) was used to visualise the chemiluminescence.

### Synthesis of HA-ta22ed Activity-Based Monoubiquitin Probes:

### Expression and Purification of HA-Ub₇₅-MeSNa

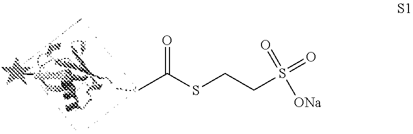

The expression and purification of HA-Ub₇₅-MeSNa was carried out according to literature procedures (S. Chong, F. B. Mersha, D. G. Comb, M. E. Scott, D. Landry, L. M. Vence, F. B. Perler, J. Benner, R. B. Kucera, C. A. Hirvonen, J. J. Pelletier, H. Paulus, M. Q. Xu, Gene 1997, 192, 271-281; A. Borodovsky, H. Ovaa, N. Kolli, T. Gan-Erdene, K. D. Wilkinson, H. L. Ploegh, B. M. Kessler, Chem Biol 2002, 9, 1149-1159). BL21 (DE3) cells transfected with a pTYB2 plasmid encoding for a HA-tagged ubiquitin75 fusion protein containing an intein domain and chitin-binding domain (HA-Ub₇₅-intein-CBD) were transferred from a glycerol stock into LB medium (8 mL) containing ampicillin (100 μg/mL) and grown for 18 h at 37° C. at 180 rpm. The cells were transferred into fresh LB medium (300 mL) containing ampicillin (100 μg/mL) and grown at 37° C. at 180 rpm until an OD₆₀₀ of 0.6 to 0.9 was reached. IPTG was added at a final concentration of 0.4 mM and the bacteria were incubated at 18° C. for 16 h with vigorous shaking. The cells were centrifuged at 8000 rpm for 15 min. The resulting pellet was re-suspended in column buffer (20 mL, 50 mM HEPES pH 6.8, 100 mM NaOAc) and lysed via sonication. The lysate was centrifuged at 14000 rpm for 45 min. A column containing chitin resin (2.5 mL) (New England Biolabs) was equilibrated with column buffer (25 mL). The clarified supernatant was run over this column. The column was washed with column buffer (25 mL). After these washes, column buffer containing sodium 2-sulfanylethanesulfonate (MeSNa) (7.5 mL; 50 mM) was run through the column before incubation in this buffer for 18 h at 37° C. with gentle shaking. HA-Ub₇₅-MeSNa was eluted in column buffer (5 mL) before concentration by spinning at 14,000 rpm in Vivaspin 500 centrifugal concentrators (Sartorious, Gottingen Germany). HA-Ub₇₅-MeSNa was desalted using a NAP-5 column (GE Healthcare, Illinois USA) and eluted in column buffer according to manufacturer's instructions. The sample was concentrated again at 14,000 rpm using Vivaspin centrifugal concentrators and the protein concentration was measured on a nanodrop (4.8 mg/mL, 100 μL).

### Coupling HA-UB₇₅-MeSNa to Bromide Warhead

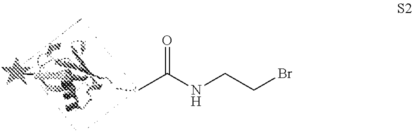

HA-Ub₇₅CH₂CH₂Br was synthesised using literature procedures (A. Borodovsky, H. Ovaa, N. Kolli, T. Gan-Erdene, K. D. Wilkinson, H. L. Ploegh, B. M. Kessler, Chem Biol 2002, 9, 1149-1159). 2-bromoethylamine.HBr (31 mg, 0.15 mmol) was dissolved in column buffer (200 μL) and the pH of the solution was adjusted to pH 8.0 by the addition of aq. NaOH (1 M). HA-Ub₇₅-MeSNa in column buffer (2.2 mg/mL, 100 μL) was added to this solution and it was shaken gently for 90 min at rt. The reaction mixture was desalted using a NAP-5 column according to manufacturer's instructions, eluted in column buffer and concentrated by centrifuging at 14,000 rpm in a Vivaspin centrifugal concentrator. The protein concentration was measured on a nanodrop (1.5 mg/mL, 100 μL).

### Coupling HA-Ub₇₅-MeSNa to Alkene Warheads

N-Hydroxysuccinimide (0.2 M, 45 μL) and Tris base (100 mM, 10 μL, pH 7.5) were added to HA-Ub₇₅-MeSNa in column buffer (1.2 mg/mL, 500 μL) and incubated for 10 min at rt. Allylamine (23 μL, 0.3 mmol) or cinnamylamine (40 mg, 0.3 mmol) was added to a solution of MeCN—H₂O (1:1, 56 μL). This solution was added to the reaction mixture and the pH was adjusted to 9.0. The reaction was incubated for 18 h at 37° C. with gentle shaking. After this time, the reaction mixture was desalted using a NAP-5 column according to manufacturer's instructions and concentrated in a Vivaspin centrifugal concentrator at 14,000 rpm. The protein concentration was measured on a nanodrop, probe 1=(3.4 mg/mL, 100 μL); probe 2=(5.6 mg/mL, 100

### In Vitro DUB Labelling:

### HEK293T Cell Lysate Preparation

A HEK293T cell pellet was lysed using glass beads. To a 100 μL cell pellet, 100 μL of glass beads were added. Homogenisation buffer (200 μL; 50 mM Tris pH 7.4, 5 mM MgCl₂, 250 mM sucrose, 1 mM DTT or 1 mM TCEP) was added. The mixture was vortexed for 20 s before being placed on ice for 90 s. This sequence was repeated 20 times. Cell debris and glass beads were pelleted by centrifuging at 14,000 rpm for 5 min. The resulting supernatant was aspirated off. The protein concentration of the clarified extract was measured by nanodrop (19.9 mg/mL, 200 μL).

In Vitro Ub₇ₛCH₂CH₂Br Probe Labelling

HA-Ub₇₅-Br probe S2 (0.75 μL, 1.5 mg/mL in column buffer) was incubated with HEK293T cell lysate (2.5 μL, 19.9 mg/mL in homogenisation buffer). The final volume of the labelling was adjusted to 30 μL with homogenisation buffer for the lysate labelling. Incubation was carried out for 90 min at 37° C. with gentle shaking. Upon completion, 2× reducing sample buffer (15 μL) was added and the proteins were heated to 95° C. for 5 min. The samples were separated using a 12% SDS-PAGE and visualised using silver staining or western blotting.

Optimised In Vitro Thiol-Ene Labelling with Alkene Probes

The relevant alkene probe (1-4 μg) was incubated with HEK293T cell lysate (2.5 μL, 19.9 mg/mL in homogenisation buffer) or OTUB1 (2 μg). The final volume of the labelling was adjusted to 30 μL with homogenate buffer containing TCEP (1 mM) for the lysate labelling, or phosphate buffer (pH 8.0) containing TCEP (1 mM) for the recombinant enzyme labelling. The probes were pre-incubated with the DUBs for 90 min at 37° C. with gentle shaking before the addition of radical initiator 2,2-dimethoxy-2-phenyl-acetophenone (DPAP) (0.25 μM) and radical stabiliser 4′-Methoyacetophenone (MAP) (0.25 μM). The reaction mixture was degassed for 2 min with N₂ and exposed to UV light (365 nm) for 2 min. 2× reducing sample buffer (30 μL) was added and the samples were heated at 95° C. for 5 min. Proteins where visualised using silver staining and western blotting after being separated on a 12% SDS-PAGE.

In Vitro Thiol-Ene Labelling with Alkene Probes and Denatured OTUB1

OTUB1 (2 μg) was denatured either by heating at 95° C. for 10 min or by the addition of SDS (0.5% final concentration). The final volume of the labelling was adjusted to 30 μL with phosphate buffer (pH 8.0) containing TCEP (1 mM). In this step SDS concentration was reduced fifteen-fold. The probes were pre-incubated with the DUBs for 90 min at 37° C. with gentle shaking before the addition of radical initiator DPAP (0.25 μM) and radical stabiliser MAP (0.25 μM). The reaction mixture was degassed for 2 min with N₂ and exposed to UV light (365 nm) for 2 min. 2× reducing sample buffer (30 μL) was added and the samples were heated at 95° C. for 5 min. Proteins where visualised using silver staining and western blotting after being separated on a 12% SDS-PAGE.

### PR-619 Pre-Incubation Assay

PR-619 was pre-incubated with HEK293T cell lysate (2.5 μL, 19.9 mg/mL) on ice for 30 min at a range of concentrations. Probe 1 (0.3 μL, 3.4 mg/mL in column buffer) was added giving the labelling a final volume of 30 μL. The reaction mixture was incubated for a further 90 min before addition of DPAP (0.25 μM) and MAP (0.25 μM) and degassing for 2 min with N₂. The mixture was exposed to UV light (365 nm) for 2 min. 2× reducing sample buffer (30 μL) was added and the samples were heated at 95° C. for 5 min.

### PR-619 Equilibrium Disruption Assay

Probe 1 (0.3 μL, 3.4 mg/mL in column buffer) or HA-Ub₇₅-Br probe S2 (0.75 μL, 1.5 mg/mL in column buffer) was incubated with HEK293T cell lysate (2.5 μL, 19.9 mg/mL) at 37° C. for 60 min. PR-619 was added at a range of concentrations and the mixture was incubated for a further 30 min at 37° C. DPAP (0.25 μM) and MAP (0.25 μM) were added and the mixture was degassing for 2 min with N₂. The mixture was exposed to UV light (365 nm) for 2 min. 2× reducing sample buffer (30 μL) was added and the samples were heated at 95° C. for 5 min.

Immunoprecipitation (IP): The relevant alkene probe (5 μg) was pre-incubated with HEK293T cell lysate (12.5 μL, 19.9 mg/mL in homogenate buffer) in NET buffer (136 μL; 50 mM Tris pH 7.5, 5 mM EDTA, 150 mM NaCl, 0.5% NP-40) containing TCEP (1 mM) for 90 min at 37° C. DPAP (0.25 μM) and MAP (0.25 μM) were added and the solution was degassed with N₂ for 2 min. The solution was exposed to UV light (365 nm) for 2 min. SDS solution (10% in dH₂O, 7.5 μL) was added to the reaction before vortexing for 30 s and sonication for 2 min. The mixture was diluted with homogenate buffer (1500 μL). EZview™ Red Anti-HA Affinity Gel (100 μL of 50% slurry) was equilibrated by adding NET buffer (750 μL), gently inverting and centrifuging at 9000 rpm. The supernatant was aspirated, and the equilibration step was repeated. The lysate was added to the equilibrated beads and incubated at 4° C. for 90 min with rolling. The mixture was centrifuged at 9000 rpm for 30 s and the supernatant was aspirated. NET buffer (750 μL) was added to the beads which were inverted until the beads were fully resuspended before being centrifuged at 9000 rpm for 30 s. This washing step was repeated four times. After the final wash glycine buffer (250 μL, 150 mM, pH 2.5) was added to the beads. The solution was inverted until the beads were resuspended and then left on ice for 1 min. The solution was centrifuged at 9000 rpm for 30 s. The resulting supernatant was aspirated, and this elution step was repeated. 1× reducing sample buffer (250 μL) was added to the beads which were heated at 95° C. for 5 min. A small % of the samples were separated by 12% SDS-PAGE and visualised by western blotting. The remainder of the samples was subject to tryptic digest using the FASP protocol, desalted by zip tipping and analysed by LC-MS/MS using an Orbitrap.

### Mass Spectrometry

### CHCl₃/MeOH Extraction

Probe samples were concentrated using a CHCl₃/MeOH extraction prior to an in-solution digest to identify the C-terminal peptide. MeOH (600 μL) and CHCl₃ (150 μL) were added to a sample of protein (200 μL) and the solution was vortexed for 20 s. dH₂O (450 μL) was added and the sample was vortexed for a further 20 s. The sample was centrifuged at 14,000 rpm for 2 min. The upper layer was aspirated off and discarded. The sample was diluted with MeOH (450 μL), vortexed for 20 s and centrifuged at 14,000 rpm for 1 min. The supernatant was aspirated and discarded. The pellet was prepared for an in-solution digestion.

### In-Solution Digest Following CHCl₃/MeOH Extraction

The protein pellet obtained using a CHCl₃/MeOH extraction was diluted in urea buffer (50 μL; 6 M urea, 33 mM Tris pH 7.8) and dissolved by vortexing for 20 s and sonicating for 2 min. The sample was diluted with dH₂O (250 μL), vortexed for 20 s and sonicated for a further 2 min. Elastase was added in a 1:15 dilution relative to the protein concentration. The digest was carried out at 37° C. with gentle shaking for 16 h. Samples were prepared for MS analysis by zip-tipping analysed by captive spray ionisation mass spectrometry.

### In-Gel Digest

Samples were separated by SDS-PAGE and visualised by silver staining. Bands of interest were excised, cut into small pieces and incubated for 18 h in wash solution (200 μL; 50% MeOH, 45% dH₂O, 5% formic acid). The wash solution was aspirated, and fresh wash solution was added. The samples were incubated for a further 2 h at rt. The wash solution was removed, and the gel pieces were dehydrated for 5 min in MeCN (2×200 μL). DTT buffer (30 μL; 100 mM NH₄HCO₃, 10 mM DTT) was added to the gel pieces and they were incubated for 30 min at rt. The DTT buffer was removed and iodoacetamide solution (30 μL, 50 mM) was added. The samples were incubated for a further 30 min. After the removal of the iodoacetamide solution the gel pieces were dehydrated for 5 min in MeCN (200 μL). Rehydration was performed in NH₃HCO₃ solution (200 μL, 100 mM). Dehydration and rehydration steps were repeated. Trypsin stock was diluted in NH₃HCO₃ solution and this 1× stock (30 μL, 20 μg/mL) was added to the dehydrated gel pieces. The solution was incubated on ice for 10 min with gentle mixing. Following this incubation step, NH₃HCO₃ solution (5 μL, 50 mM) was added to the mixture and it was incubated for 18 h at 37° C. with gentle shaking. After this incubation, NH₃HCO₃ solution (50 μL, 50 mM) was added. The gel pieces were incubated in this mixture for 10 min with occasional vortexing. The supernatant was transferred to a fresh microcentrifuge tube. Extraction buffer 1 (50 μL; 50% MeCN, 45% dH₂O, 5% formic acid) was added to the gel pieces. The pieces were incubated for 10 min in this buffer with occasional vortexing. The supernatant was then added to the collection tube and the gel pieces were incubated for a further 10 min in extraction buffer 2 with periodic vortexing (85% MeCN, 10% dH₂O, 5% formic acid). The supernatant was again added to the collection tube. For bigger protein bands an additional extraction with extraction buffer 2 was performed. The combined supernatants were dried in a vacuum centrifuge, resuspended in buffer A (20 μL; 98% dH₂O, 2% MeCN, 0.1% formic acid) and analysed by captive spray ionisation mass spectrometry.

### Filter Aided Sample Preparation (FASP)

FASP (L. L. Manza, S. L. Stamer, A. J. Ham, S. G. Codreanu, D. C. Liebler, Proteomics 2005, 5, 1742-1745) was carried out using Vivaspin 500 centrifugal concentrators (10,000 MWCO). The concentrator was conditioned with 50 μL dH₂O. It was spun at 11800 rpm for 2 min. The protein solution to be digested was transferred to the filter and centrifuged at 13000 rpm until concentrated to a maximum of 25 μL. UA buffer (200 μL, 8 M urea in 0.1 M Tris/HCl pH 8.5) was added to the filter and centrifuged at 11800 rpm for 15 min. This step was repeated twice. DTT solution (100 μL, 10 mM in UA buffer) was added to the concentrator and vortexed for 5 s. It was centrifuged at 11800 rpm for 15 min. IAA solution (100 μL, 50 mM) was added and the solution was vortexed for 5 s and then centrifuged again at 11800 rpm for 15 min. Washes were performed using UA buffer (3×100 μL) followed by NH₄HCO₃ solution (3×100 μL, 50 mM). After the final wash, trypsin solution (200 μL, 50 mM NH₄HCO₃ solution, 1:50 enzyme:protein) was added and the concentrator was incubated overnight at 37° C. The concentrator was centrifuged at 11800 rpm for 15 min. NaCl solution (50 μL, 0.5 M) was added and it was centrifuged at 11800 rpm until all the of the solution had passed the filter. Samples were de-salted by zip-tipping and analysed by LC-MS/MS.

### Zip-Tip Purification

A zip-tip (Merk Millipore, Massachusetts USA) was equilibrated by aspirating and dispensing buffer B (for peptides: 80% MeCN, 20% H₂O, 0.1% TFA; for full proteins; 65% MeCN, 35% H₂O, 0.1% TFA) four times and further four times with buffer A (2% MeCN, 98% H₂O, 0.1% TFA). The protein sample was aspirated across the tip ten times. Buffer A was used to wash the sample by aspirating and dispensing four times. The protein or peptides were then eluted in buffer B (2×10 μL) and dried using a vacuum centrifuge. The sample was analysed by MALDI or LC-MS/MS.

### MALDI-TOF MS

MALDI-TOF analysis was carried out on a BRUKER Ultraflextreme MALDI-TOF/TOF mass spectrometer. The matrix used was a saturated solution of HCCA (α-Cyano-4-hydroxycinnamic acid) in TA 85% (85% ACN with 0.1% TFA), and the calibrant was prepared in the same matrix. The matrix (1 μL) was mixed with the sample (1 μL) and 1 μL of this mixture was deposited onto a ground steel MALDI target plate and allowed to dry in air. Mass spectra were recorded in positive reflection mode.

### Orbitrap Mass Spectrometry

Protein digests were redissolved in 0.1% TFA (30 μL per sample) by agitation (1200 rpm, 15 min) and sonication in an ultrasonic water bath (10 min). This was followed by centrifugation (14,000 rpm, 5° C., 10 min) and transfer to MS sample vials. LC-MS/MS analysis was carried out in technical duplicates (4.0 μL per injection) and separation was performed using an Ultimate 3000 RSLC nano liquid chromatography system (Thermo Scientific) coupled to a Orbitrap Velos mass spectrometer (Thermo Scientific) via an Easy-Spray nano-electrospray source (Thermo Scientific). Samples were injected and loaded onto a trap column (Acclaim PepMap 100 C18, 100 μm×2 cm) for desalting and concentration at 8 μL/min in 2% acetonitrile, 0.1% TFA. Peptides were then eluted on-line to an analytical column (Acclaim Pepmap RSLC C18, 75 μm×50 cm) at a flow rate of 250 nL/min. Peptides were separated using a 120 min gradient, 4-25% of buffer A for 90 min followed by 25-45% buffer B for another 30 min (buffer A: 5% DMSO, 0.1% FA; buffer B: 75% acetonitrile, 5% DMSO, 0.1% FA) and subsequent column conditioning and equilibration. Eluted peptides were analysed by the mass spectrometer operating in positive polarity using a data-dependent acquisition mode. Ions for fragmentation were determined from an initial MS1 survey scan at 30,000 resolution, followed by CID (Collision-Induced Dissociation) of the top 10 most abundant ions. MS1 and MS2 scan AGC targets were set to 1⁶ and 3⁴ for maximum injection times of 500 ms and 100 ms respectively. A survey scan m/z range of 350-1500 was used, normalised collision energy set to 35%, charge state screening enabled with +1 charge state rejected and minimal fragmentation trigger signal threshold of 500 counts. Data was processed using the MaxQuant^{[39]} software platform (v1.6.7.0), with database searches carried out by the in-built Andromeda search engine against the Swissprot H. sapiens database (version 20180104, number of entries: 20,244). A reverse decoy database approach was used at a 1% false discovery rate (FDR) for peptide spectrum matches. Search parameters included: maximum missed cleavages set to 3, fixed modification of cysteine carbamidomethylation and variable modifications of methionine oxidation, asparagine deamidiation and protein N-terminal acetylation. Label-free quantification was enabled with an LFQ minimum ratio count of 1.

### Captive Spray Ionisation Mass Spectrometry

Captive spray ionisation was performed using a Thermo Scientific UltiMate 3000RSLCnano LC (Waltham, Mass. USA) equipped with an Acclaim PepMap C18 (2 μm, 0.075 mm×150 mm) column. For each injection, 5 μL of a (1 μg/μL) digested peptide was loaded onto a Nano Trap Column (100 μm I.D.×2 cm, packed with Acclaim PepMap100 C18) at 10 μL/min with 95% water/5% acetonitrile/0.1% formic acid for 3 min. Trapped peptides were eluted onto the analytical column using a multi-step gradient with a flow rate of 0.3 μL/min. The gradient utilised two mobile phase solutions: A, water/0.1% formic acid and B, acetonitrile: 0 min, A (98%), B (2%); 3 min, A (98%), B (2%); 63 min A (65%), B (35%); 64 min A (5%), B (95%); 66 min A (5%), B (95%); 67 min A (98%), B (2%); 75 min A (98%), B (2%). Peptide digest were analysed on a Bruker compact Qq-TOF mass spectrometer via CaptiveSpray nanoBooster (Bremen Germany). Precursor ions were scanned from 150 m/z to 2200 m/z at 2 Hz with a cycle time of 3.0 seconds, with fixed windows excluded (20-350, 1221-1225, 2200-40000). Smart Exclusion was used to ensure only chromatographic peaks were selected as precursors. Active Exclusion enabled the analysis of less-abundant ions to be analysed and not excluded from precursor selection. Data acquired on the Bruker compact was converted to mzXML format and searched against a custom database containing the probe sequence inserted into a uniprot database with taxonomy restricted to human on Peptide Shaker.

General chemical methods: and ¹³C NMR spectra were recorded on Bruker 400 MHz or 600 MHz system spectrometers. Spectra were recorded in DMSO-d₆ or CDCl₃ relative to residual DMSO (6=2.50 ppm) or CHCl₃ (δ=7.26 ppm). Chemical shifts are reported in parts per million (ppm), coupling constants are reported in Hertz (Hz) and are accurate to 0.2 Hz. NMR spectra were assigned using HSQC and HMBC experiments. Mass spectrometry measurements were carried out on a Bruker ESI or APCI HRMS. Melting points were measured using a Griffin melting points apparatus and are uncorrected. Infrared (IR) spectra were obtained on a Perkin Elmer spectrophotometer. Flash column chromatography was carried out using silica gel, particle size 0.04-0.063 mm. TLC analysis was performed on precoated 60F₂₅₄ slides and visualised by UV irradiation, potassium permanganate stain (3 g KMnO₄, 20 g K₂CO₃, 300 mL dH₂O) and ninhydrin stain (1.5 g ninhydrin, 5 mL, AcOH, 500 mL EtOH 95%). All solvents were obtained from commercial sources and used as received. Petroleum ether refers to the fraction of petroleum ether that boils at 40-60° C.

### Synthesis of (E)-1-phenyl-3-phthalimido-2-propene

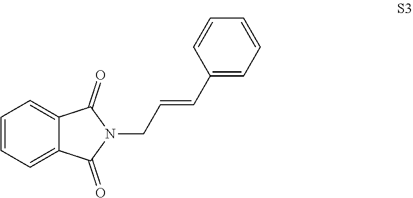 [SMILES: O=C1c2ccccc2C(=O)N1C/C=C/c1ccccc1]

Cinnamyl bromide (500 mg, 2.54 mmol) and potassium phthalimide (729 mg, 3.94 mmol) were dissolved in dry DMF (10 mL) under argon. The reaction mixture was stirred at rt for 3 h. TLC analysis (petroleum ether) showed complete consumption of cinnamyl bromide (R_{f}=0.6) and formation of the product (R_{f}=0.1) after this time. The solution was diluted with Et₂O (40 mL) and brine (30 mL) and the white precipitate formed was collected by vacuum filtration. The aqueous layer was extracted with Et₂O (2×30 mL). The combined organic layers were dried over MgSO₄, filtered and concentrated to afford the crude product as a yellow solid. This was combined with the precipitated product and recrystallised from toluene to afford the product S3 as colourless crystals (411 mg, 62%); mp 152-154° C. (toluene). Lit. 154° C.-155° C.

The spectroscopic data (not shown) was in agreement with those reported in the literature.

### Synthesis of (E)-3-phenyl-prop-2-en-1-amine

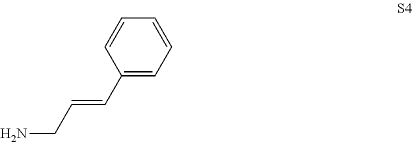

(E)-1-phenyl-3-phthalimido-2-propene S3 (700 mg, 2.66 mmol) was dissolved in MeOH (12 mL). Hydrazine hydrate solution (80%, 150 μL, 2.95 mmol) was added dropwise and the reaction was stirred at rt for 2 h. TLC analysis after this time showed the complete consumption of the starting material (petroleum ether-EtOAc, 3:1; R_{f}=0.8) and formation of the product S4 (H₂O-IPA-EtOAc, 1:2:2; R_{f}=0.2). The reaction was cooled to 4° C. resulting in the formation of a white precipitate. The white precipitate was isolated by vacuum filtration and washed with MeOH (3×10 mL). The filtrate was concentrated under reduced pressure and the residue was dissolved in DCM (20 mL) and aq. KOH (20 mL). The aqueous layer was extracted with DCM (3×20 mL) and the combined organic layers were concentrated to afford the product S4 as a yellow oil (228 mg, 65%).

The spectroscopic data (not shown) was in agreement with those reported in the literature.

### Optimised Eosin Y Recombinant Enzyme Labelling

Probe 1 (2 μg) was incubated with OTUB1 (0.2 μL, 13.78 μg/μL, in storage buffer) or UCHL1 (3.3 μL, 0.9 μg/μL, in storage buffer). The final volume of the labelling was adjusted to 30 μL with homogenisation buffer containing TCEP (1 mM). A stock solution of Eosin Y (0.29 mM in DMSO—homogenisation buffer, 4:1) was prepared fresh before use and protected from light. The reaction was preincubated for 90 min at 37° C. with gentle shaking before the addition of Eosin Y (0.5 μL of stock for recombinant enzyme labelling, final conc.=5 μM). Samples were exposed to white light (10 W) from 50 cm for 5 min or ambient light for 30 min at 37° C. Upon completion, 2× reducing sample buffer (30 μL) was added and the proteins were heated to 95° C. for 5 min. Proteins where visualised using silver staining and anti-HA western blotting after being separated on a 12% SDS-PAGE.

1. A process for labelling a target protein, the process comprising
providing a probe-protein complex comprising a probe and the target protein;
the probe comprising a recognition element and a warhead;
the target protein comprising a cysteine residue and a recognition site;
wherein the recognition element is reversibly bound to the recognition site; and
applying a stimulus to induce a radical reaction in the probe-protein complex to covalently bond the warhead to the cysteine residue, thereby labelling the target protein.

2. The process of claim 1, wherein applying a stimulus comprises exposing the probe-protein complex to light.

3. The process of claim 1, wherein applying a stimulus comprises employing one or more radical initiators.

4. The process of claim 3, wherein the one or more radical initiators are selected from acetophenones, azo compounds and/or organic peroxides.

5. The process of claim 1, wherein applying a stimulus comprises employing
(i) 2,2-dimethoxy-2-phenylacetophenone (DPAP); or
(ii) 2,2-dimethoxy-2-phenylacetophenone (DPAP) with 4′-methoyacetophenone (MAP); or
(iii) bismuth oxide; or
(iv) eosin.

6. The process of claim 1, wherein providing the probe-protein complex comprises incubating the target protein together with the probe for 5 minutes or more, prior to the stimulus being applied.

7. The process of claim 1, wherein
(i) the probe comprises a tag; or
(ii) the process comprises a further step of tagging the labelled protein.

8. The process of claim 1, wherein the target protein comprises an enzyme.

9. The process of claim 8, wherein the target protein is selected from a cysteine protease, a glycosidase, a kinase, a phosphatase, an isomerase, an oxidoreductase, a hydrolase, a thiolase, a sulfurtransferase, or a synthase.

10. The process of claim 8, wherein the enzyme comprises a deubiquitinating enzyme (DUB).

11. The process of claim 1, wherein the target protein comprises a deubiquitinating enzyme (DUB) and the recognition element comprises ubiquitin.

12. The process of claim 1, wherein the warhead comprises an alkene moiety; or a strained ring system; or an internal alkyne.

13. The process of claim 1, wherein the warhead comprises an alkenyl group having the general structure I 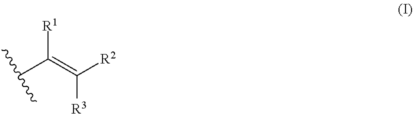
wherein each of R¹, R² and R³ is independently selected from H, NH₂, an alkyl group, a further alkenyl group, an aryl group and an aralkyl group.

14. The process of claim 13, wherein R¹ is H or an alkyl group.

15. The process of claim 13, wherein R² is H or an alkyl group.

16. The process of claim 13, wherein R³ is H or an alkyl group.

17. The process of claim 1, wherein the recognition element has an amino acid sequence and the warhead is attached at the carboxy terminus of said amino acid sequence.

18. The process of claim 17 wherein the warhead comprises an alkenyl group and the alkenyl group is attached to the carboxy terminus of said amino acid as shown in the general structure (VII) 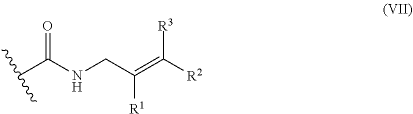
wherein each of R¹, R² and R³ is independently selected from H, an alkyl group, a further alkenyl group, an aryl group and an aralkyl group.

19. A probe-protein complex comprising a probe and a target protein;
the probe comprising a recognition element and a warhead; and
the target protein comprising a cysteine residue and a recognition site;
wherein the recognition element is reversibly bound to the recognition element.

20. A probe comprising a tag, a recognition element and a warhead,
wherein the warhead is capable of a radical reaction with a cysteine residue to covalently bond the warhead to the cysteine residue.
